(19) United States
(12) Patent Application Publication
(10) Pub. No.: US 2011/0071563 A1
(43) Pub. Date: Mar. 24, 2011

(54) LAMINECTOMY FORCEPS

(76) Inventor: Vincenzo Magliani, Teramo (IT)

(21) Appl. No.: 12/736,869

(22) PCT Filed: May 18, 2009
(86) PCT No.: PCT/EP2009/056020

§ 371 Date: Nov. 18, 2010

(30) Foreign Application Priority Data
May 19, 2008 (IT) MC2008A000080

(51) Int. Cl. A61B 17/28

(52) U.S. Cl. 606/205

(57) ABSTRACT

The present invention relates to a laminectomy forceps (1) that is provided with a lever mechanism composed of at least two, preferably three arms (8, 9, 10) that multiplies the force exerted by the surgeon on the actuation lever (5) of the forceps.

The present invention relates to a laminectomy forceps as claimed in the preamble of the first claim.

Laminectomy is a surgical operation that provides for total or partial removal of one or more laminae of one or more vertebrae, opening the vertebral canal, to treat spinal pathologies of different nature; laminectomy is used to obtain the excision of the posterior arch of the vertebra.

During laminectomy the surgeon removes bone fragments with a forceps: different types of laminectomy forceps are known at the state of the art, such as Kerrison forceps or Citelli forceps.

In general, all forceps are provided with a handle joined to a fixed beak, with a sliding tray actuated by a lever in opposite position to the handle; the beak has a backing end for the cutting end of the tray in order to generate the cutting area.

The actuation lever is articulated to the tray according to a first-degree lever; more precisely, a pivot is positioned between the handle and the actuation lever and the prosecution of the handle lever, beyond the pivot, towards the tray, is the actuation arm for the tray; the tray is normally provided with an overturned U cross-section, in such a way that an empty area is obtained between the tray and the beak; the handle rests on the palm of the surgeon, who actuates the actuation lever with his fingers.

The bone excision operation is performed in multiple phases, and in each phase the surgeon actuates the forceps to remove a fragment; obviously, this operation must be extremely precise, also because the operation area is dangerously close to the nerve centres of the vertebral column, which could be damaged with pernicious consequences.

The laminectomy forceps of known type are impaired by several drawbacks: firstly, the force exerted by the surgeon on the actuation lever to remove every bone fragment is relatively high and causes early fatigue of the surgeon's hand.

Secondly, the bone fragments removed from the vertebrae get often stuck between the tray and the beak, inside the overturned-U section of the tray, in such a way that their removal slows down the surgical operation and sometimes the forceps must be changed several times during the same surgical phase.

Thirdly, the cutting end of the tray and the cutting ends of the backing border of the beak tend to wear out rapidly, requiring the early replacement of the entire forceps.

The purpose of the present invention is to solve the aforementioned inconveniences with a surgical forceps for laminectomy as claimed in the first claim.

Further advantageous characteristics are the subject of the enclosed claims.

Advantageously, the laminectomy forceps according to the present invention provides for multiplying the force exerted by the surgeon on the actuation lever by means of a lever mechanism composed of at least two, preferably three arms, in such a way to prevent the surgeon's early fatigue, and increase the speed and accuracy of the surgeon's action.

Secondly, the forceps according to the present invention comprises a cleaning device with sliding rod inside the tray, in such a way to remove the bone fragments that get stuck in the tray.

Thirdly, the forceps as claimed in the present invention allows for disassembling the cutting parts when they are worn out in order to replace them when necessary, without changing the entire forceps.

Additional characteristics and advantages of the present invention will appear more evident from the following description and enclosed drawings, which have only an illustrative, not limiting purpose, wherein:

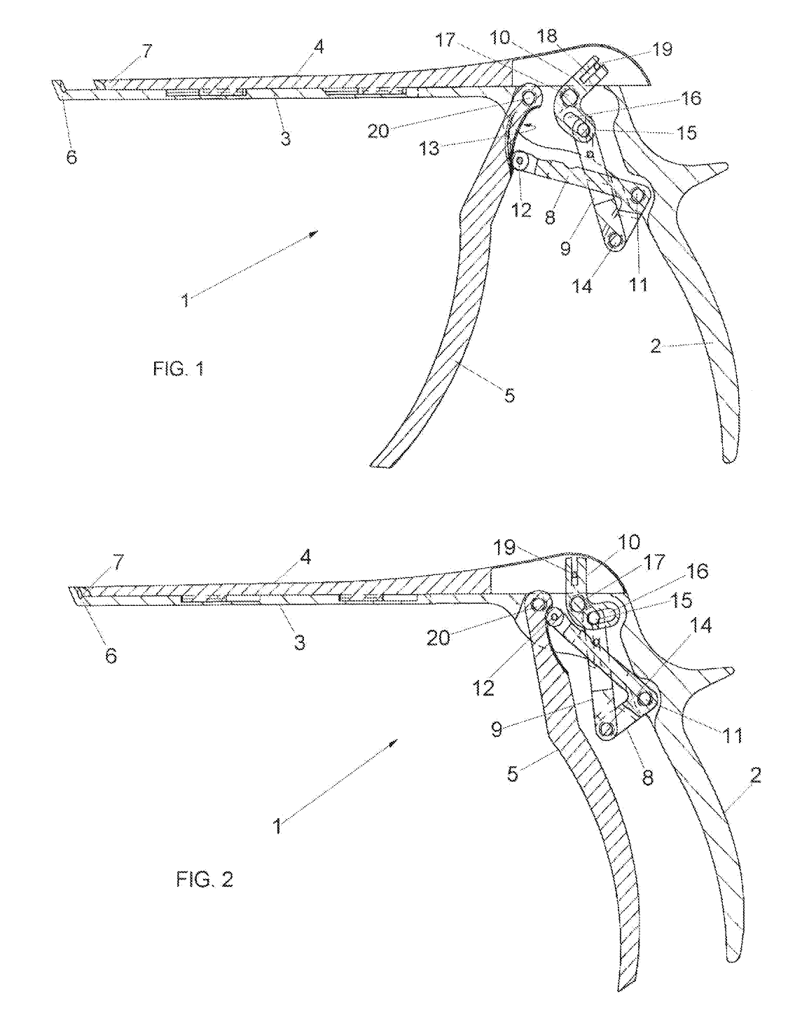

FIG. 1 is a sectional view of a laminectomy forceps according to the present invention in open condition;

FIG. 1 is a sectional view of a laminectomy forceps according to the present invention in closed condition;

FIG. 1 is a lateral view of a laminectomy forceps according to the present invention in open condition;

FIG. 1 is a lateral view of a laminectomy forceps according to the present invention in closed condition;

FIG. 5 is a sectional view of the tray of the laminectomy forceps according to the present invention provided with cleaning device;

FIG. 6 is a top view of the tray of the laminectomy forceps according to the present invention provided with cleaning device;

FIG. 7 is a cross-sectional view of the tray of the laminectomy forceps according to the present invention provided with cleaning device;

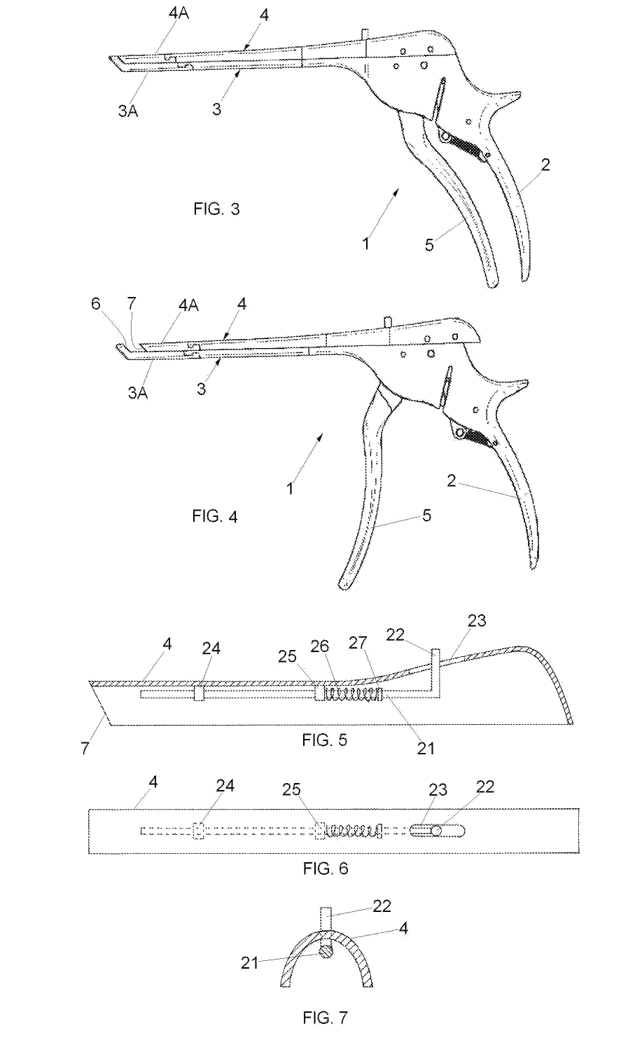

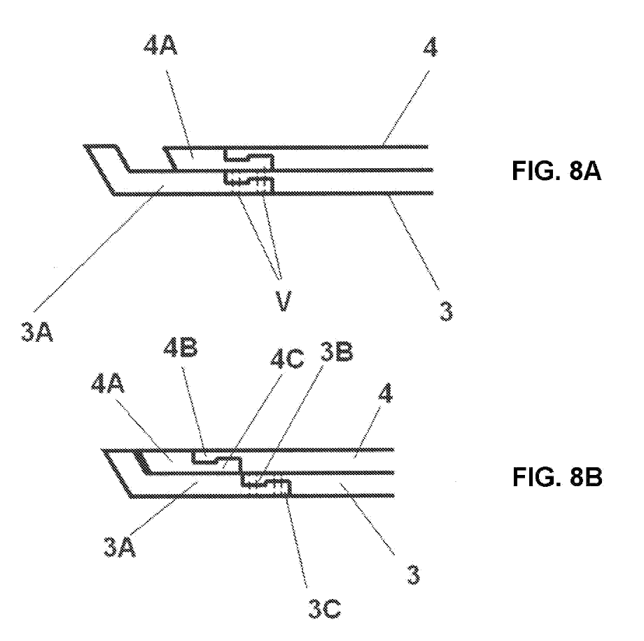

FIGS. 8A and 8B are enlarged views of FIG. 3 and FIG. 4.

With reference to FIGS. 1 and 2, the laminectomy forceps (1) according to the present invention comprises: a handle (2) joined to a fixed beak (3) with a sliding tray (4) actuated by an actuation lever (5) in opposite position to the handle (2), in such a way that the surgeon places the handle (2) on his palm and actuates the actuation lever (5) with the fingers of the same hand.

The fixed beak (3) is provided with a backing end (6) that acts as stop for the cutting end (7) of the tray (4), in such a way to generate the cutting area in which the surgeon places the part of bone to be removed.

The forward movement of the tray (4) towards the backing end (6) determines the cut and excision of the bone fragment included between them by means of the cutting edges situated in opposite position on the cutting end (7) and the backing end (6).

The actuation lever (5) according to the precepts of the present invention is articulated with the tray (4) by means of a lever mechanism designed to multiply the force exerted by the surgeon on the actuation lever (5) and transmit the multiplied force to the tray (4).

According to the preferred embodiment illustrated in the enclosed figures the mechanism lever is composed of three arms: a first arm (8) hinged on the handle (2), which is articulated to a second arm (9), which is in turn articulated with a third arm (10) that actuates the tray (4), as explained below.

The first arm (8) is hinged at one end to the handle (2) with the first pin (11) and is provided at the opposite end with a roll (12) free to slide in the inlet (13) obtained on the internal side of the actuation lever (5): further to the forward movement of the actuation lever (5) towards the handle (2), the roll (12) moves in the inlet between two stop positions, the first one illustrated in FIG. 1 and the second one in FIG. 2, thus determining the rotation of the first arm (8) around the first pin (11).

The first arm (8) has an L-shape and its free end in opposite position to the roll (12) is hinged with the second pin (14) to the second arm (9), thus moving it consequently.

The free end of the second arm (9) opposite to the end hinged with the first arm (8) is provided with a third pin (15) that slides inside the slot (16) of the third arm (10), which has an L-shape and is hinged to the handle (2) with the fourth pin (17).

The free end of the third arm (10) in opposite position to the slot (16) is provided with a sliding guide (18) for the constraint (19), which is joined with the tray (4) that can only slide forward and backward on the fixed beak (3).

Obviously, also the actuation lever (5) is hinged on the handle (2) by means of the pin (20).

The operation of the forceps (1) according to the present invention is shown in FIGS. 1 and 2, which illustrate the two extreme conditions of open and closed forceps: when the surgeon actuates the actuation lever (5) by moving it closer to the handle (2), the roll (12) slides on the inlet (13) and the first arm (8) rotates around the first pin (11); the opposite end of the first arm (8) actuates by means of the second pin (14) on the second arm (9) that moves, making the third pin (15) slide inside the slot (16), thus actuating the third arm (10) in rotation around the fourth pin (17); the fourth arm actuates on the constraint (19) by means of the sliding guide (18) in which the constraint (19) moves, driving the tray (4) closer to the cutting end (7) towards the backing end (6).

In the aforementioned configuration the lever mechanism used to actuate the tray (4) comprises two arms (8, 10) that are both hinged on the handle (2) and mutually connected by an additional arm (9).

The following calculation is given for merely illustrative purposes: with the following dimensions:

length of actuation lever (5)=115.86 mm=A;

distance between the axis of the actuation pin (20) and the axis of the roll (12) with forceps in closed condition=3.21 mm=B;

distance between the axis of the first pin (11) and the axis of the third pin (15) with forceps in closed condition=26.05 mm=C;

distance between the axis of the first pin (11) and the axis of the second pin (14) with forceps in closed condition=15.43 mm=D;

distance between the axis of the second pin (14) and the axis of the fourth pin (17) with forceps in closed condition=39.63 mm=E;

a load F₁=5 kg applied at the end of the actuation lever (5) determines a load F₂ exerted on the tray by the constraint (19) equal to:

F₂=F₁*(A/B*C/D*E/F)=1280 kg.

The above clearly shows the advantages of the said lever mechanism, since the force transmitted by the handle lever to the tray can be hardly obtained using the principle of first degree lever, such in the case of the known forceps, in which the forceps should be dimensioned in such a way to prevent the correct use in the surgical field and/or use with two hands.

With the forceps according to the present invention the surgeon can advantageously avoid early fatigue of the hand, dosing the force correctly and being more accurate in his action.

Alternatively, although with a lower force ratio, the third arm (10) can be eliminated and the second arm (9) can be extended until it actuates on the constraint (19) of the tray.

An additional advantageous characteristic of the forceps according to the present invention is illustrated in FIGS. 3 and 4, in which both the tool-holder portion (3A) of the beak (3) with the cutting backing end (6) and the tool-holder portion (4A) of the tray (4) with the cutting end (7) are of removable type.

Basically, the tool-holder portion (3A) of the fixed beak (3) is a separate part of the beak (3) and is joined to the beak (3) by means of screws or similar removable connection means that allow for replacement when the cutting edges are worn out.

Similarly, also the tool-holder portion (4A) of the tray (4) is a separate part of the tray (4) and is joined to the tray (4) by means of screws or similar removable connection means that allow for replacement when the cutting edges are worn out.

Advantageously, this allows for replacing only the cutting edges when they have lost sharpening due to extended use, without having to replace the entire forceps, as in the case of known models of forceps.

According to a preferred embodiment (shown in FIGS. 8A and 8B), the tool-holder portions (4A, 3A) are engaged on the tray (4) and on the fixed beak (3), respectively, as illustrated, with double-hook engagement means (3B, 3C and 4B, 4C) for rapid assembly; in this case, a simple analysis of the figures demonstrates the operation principle: when the forceps is in disassembled condition (that is to say without the tool-holder end 4A, 3A), the forceps is actuated in closed condition, in such a way that the tray (4) moves forward until the section with downward-facing hooked profile (4b) protrudes from the fixed beak (3), and then the tool-holder portion (4A) is inserted and the actuation lever is released, making the tray (4) move backwards towards the handle (2).

This causes the locking in position of the tool-holder portion (4A) also without screws and without positioning the tool-holder portion (3A) of the fixed beak (3): when the forceps is opened, the tool-holder portion (4A) rests under the fixed beak (3) and is prevented on top from being released from the double-hook profile (4B and 4C).

Then the forceps is completed by positioning the tool-holder portion of the fixed beak (3A) in the double-hook seat (3B and 3C) and by fixing it to the fixed beak (3) with screws.

Not only this allows for replacing the cutting edges when they are worn out, but also to replace them quickly, since they are both fixed in operating position with only one screw.

FIGS. 5, 6 and 7 illustrate an additional advantageous characteristic of the present invention, that is to say the cleaning device.

The cleaning device is basically composed of a rod (21) that slides inside the tray (4), which is provided with overturned U cross-section.

The rod can be actuated manually with a control handle (22), which is shown in an illustrative form as a transversal extension of the rod (21), sliding in a slot (23) obtained in upper position on the body of the tray (4).

The rod (21) slides inside the tray (4) in such a way to move forward towards the cutting end (7) and free the tray (4) from the bone fragments that can be stuck therein; the rod (21) slides in a sliding guide that is illustratively shown as obtained with two sliding eyelets (24, 25).

According to another advantageous characteristic the rod (21) is provided with autonomous return means, such as a spring (27) that actuates on an eyelet (25) and on a projecting ridge (27) of the rod (21).

Additional variations and additions to the forceps according to the present invention can be provided without leaving the precepts and the purpose of the present invention.

For example, automatic return means to open position of the forceps can be provided, such as a spring, either helicoidal or obtained with two pivoting metal plates in opposite position (being one fixed to the actuation lever and the other one to the handle), designed to push the actuation lever firmly away from the handle.

Moreover, autonomous return means of the cleaning rod that differ from the ones illustrated and described herein can be provided, or alternatively the sliding guide of the rod can have different shapes.

Although according to the illustrated forceps the cutting end (7) and the relevant backing end (6) are in oblique position with respect to the sliding direction of the tray (4), it is possible to provide them in perpendicular position.

An additional alternative embodiment, which is not illustrated in the enclosed figures, provides that the relative positions of the tray and the fixed beak are reversed, in such a way that the cutting area faces downwards instead of upwards, as shown.

The said alternative can be useful when the point of the operation is especially troublesome or not easily accessible with a forceps as the illustrated one.

In such a case, by applying the aforementioned precepts, the mutual positions of the tray (4) and the fixed beak (3) can be exchanged, in such a way that the fixed beak (3) is situated above the tray (4), which faces the actuation lever (5).

1. Laminectomy forceps (1) comprising a handle (2) joined to a fixed beak (3) with a sliding tray (4) actuated by an actuation lever (5) in opposite position to the handle (2), in which the fixed beak (3) is provided with a cutting backing end (6) for a cutting end (7) of the tray (4) in order to generate a cutting area and in which the actuation lever (5) is articulated with the tray in such a way to determine sliding,
forceps characterised in that
the actuation lever (5) is articulated to the tray (4) with a force multiplier lever mechanism comprising at least two (8, 10), preferably three arms (8, 9, 10).

2. Forceps as claimed in claim 1, characterised in that the actuation lever (5) actuates a first arm (8) hinged to the handle (2), the first arm (8) being articulated to a second arm (9), the second arm (9) being articulated to a third arm (10), the third arm (10) being hinged to the handle (2) and actuating the tray (4) and in which the first arm (8) and the third arm have an L shape.

3. Forceps as claimed in claim 2, characterised in that the first arm (8) is hinged at one end to the handle (2) by means of a first pin (11) and is provided at the opposite end with a roll (12) free to slide in the inlet (13) obtained on the internal side of the actuation lever (5), and in which the free end of the first arm (8) opposite to the end with the roll (12) is hinged with a second pin (14) to the second arm (9) with free end opposite to the end hinged to the first arm (8), comprising a third pin (15) that slides inside a slot (16) provided on the third arm (10) and in which the free end of the third arm (10) in opposite position to the slot (16), is provided with a sliding guide (18) for a constraint (19) joined to the tray (4) in order to make the tray (4) slide with respect to the fixed beak (3).

4. Forceps as claimed in claim 1 or 2 or 3, characterised in that the actuation lever (5) is hinged on the handle (2) by means of an actuation pin (20).

5. Forceps as claimed in any of the above claims, characterised in that the fixed beak (3) comprises a tool-holder portion (3A) with the cutting backing end (6), the said tool-holder portion (3A) being removably fixed with respect to the fixed beak (3) and in that the tray (4) comprises a tool-holder portion (4A) with the cutting end (7), the said tool-holder portion (4A) being removably fixed with respect to the tray (4).

6. Forceps as claimed in claim 5 characterised in that the tool-holder portion (3A) of the fixed beak is fixed removably by means of detachable connection means, such as screws or similar means.

7. Forceps as claimed in claims 5 or 6, characterised in that the tool-holder portions (4A, 3A) are respectively engaged on the tray (4) and on the fixed beak (3) with double-hook engagement means (3B, 3C and 4B, 4C), with the tray (4) protruding from the fixed beak (3) when the forceps is closed and the tool-holder portions (3A and 4A) are disassembled, in such a way to lock in position the tool-holder portion (4A) of the tray when the forceps is open without assembling the tool-holder portion (3A) of the fixed beak (3).

8. Forceps as claimed in one or more of the above claims characterised in that it comprises a cleaning device to clean the tray (4).

9. Forceps as claimed in claim 8, characterised in that the tray (4) has an overturned U cross-section and the cleaning device comprises a rod (21) that slides inside the tray (4).

10. Forceps as claimed in claim 9, characterised in that the rod (21) is actuated by means of a control handle (22) in transversal position to the rod (21), with the control handle (22) sliding in a slot (23) obtained in upper position on the body of the tray (4).

11. Forceps as claimed in claim 8 or 9, characterised in that the rod (21) slides inside a sliding guide that comprises t least one sliding eyelet (24, 25).

12. Forceps as claimed in one or more of claims 8 to 11 characterised in that the rod (21) is provided with autonomous return means, such as a spring (27) or similar means.

13. Forceps as claimed in one or more of the above claims, characterised in that it also comprises automatic return means to open position of the forceps, such as a helicoidal spring or two pivoting metal plates in opposite position (being one fixed to the actuation lever and the other one to the handle), designed to push the actuation lever firmly away from the handle.
